(19) United States
(12) Patent Application Publication
(10) Pub. No.: US 2022/0211294 A1
(43) Pub. Date: Jul. 7, 2022

(54) COMMUNICATION DEVICE AND COMMUNICATION SYSTEM FOR MONITORING BREATHING

(71) Applicant: Dräger Safety AG & Co. KGaA, Lübeck (DE)

(72) Inventors: Mohamed Houssem HARBI, Lübeck (DE); Timo TRALAU, Lübeck (DE); Michael BRODERSEN, Lübeck (DE); Hauke KASTENS, Lübeck (DE)

(21) Appl. No.: 17/568,232

(22) Filed: Jan. 4, 2022

(30) Foreign Application Priority Data
Jan. 5, 2021 (DE) 102021100061.0

(51) Int. Cl. A61B 5/08; A61B 5/00; A62B 18/08

(52) U.S. Cl. CPC A61B 5/0816; A62B 18/08; A61B 5/0002; A61B 5/746

(57) ABSTRACT

A communication device (100), for monitoring breathing, includes a microphone unit (110), a monitoring unit (120) and an output unit (130). The microphone unit receives an acoustic signal (105), which indicates the breath sounds (350) of a communication device user (102), from a surrounding area (112) of the microphone unit, and converts same into a digital input signal (114). The monitoring unit is configured to receive the digital input signal and to split the indicated breath sounds into individual breaths (124) based on an amplitude- and/or frequency-dependent pattern recognition (122) and to determine a respective breath duration (125) of a breath. The monitoring unit triggers a breath alarm state (126) of the communication device as a function of a consecutive sequence of breath durations. The output unit is configured to output an alarm output (132) indicating the breath alarm state after a triggering of the breath alarm state.

### CROSS REFERENCE TO RELATED APPLICATIONS

This application claims the benefit of priority under 35 U.S.C. § 119 of German Application 102021100061.0, filed Jan. 5, 2021, the entire contents of which are incorporated herein by reference.

### TECHNICAL FIELD

The present invention pertains to a communication device for monitoring breathing. Furthermore, the present invention pertains to a communication system with a plurality of communication devices according to the present invention, to a gas mask with a communication device and to a helmet, and especially to a firefighter helmet, with a communication device. Finally, the present invention pertains to a process for monitoring breathing.

### TECHNICAL BACKGROUND

It is known to make possible a communication between firefighters via a gas mask communication system. To this end, the processing of speech signals is also common in order to muffle especially high-frequency speech components and as a result to improve the intelligibility of outputted speech signals. A headset function is often additionally integrated into such a communication system for tactical radio. An analog or digital radio device is for this purpose integrated into the system with a cable or with a wireless interface.

In addition to sensors to be worn on the body, so-called dead man warnings are also known for the detection of the vital functions of a respirator user. Such a dead man warning responds, for example, the absence of movement and partly to a horizontal body position of the respirator user. If the respirator user remains motionless for a certain time, an alarm is triggered, as a result of which responders may locate the unconscious person in a simple manner.

### SUMMARY

An object of the present invention is to provide an especially advantageous communication device, especially a communication device with especially reliable detection of a present breath alarm state.

According to a first aspect of the present invention, a communication device for monitoring breathing, with a microphone unit, with a monitoring unit, and with an output unit, is provided for accomplishing this object.

The microphone unit is configured to receive an acoustic signal, which indicates the breath sounds of a communication device user of the communication device, from a surrounding area of the microphone unit, and to convert the acoustic signal into a digital input signal.

The monitoring unit is configured to receive the digital input signal and to split the indicated breath sounds into individual breaths on the basis of an amplitude-dependent pattern recognition and/or frequency-dependent pattern recognition and to determine a particular breath duration of a breath, wherein the monitoring unit is further configured to trigger a breath alarm state of the communication device as a function of a consecutive sequence of breath durations. The monitoring unit comprises one or more processors and a memory.

The output unit is configured to output an alarm output indicating the breath alarm state after a triggering of the breath alarm state.

It was found within the framework of the present invention that a microphone unit, as it is present, for example, within the framework of a communication system as well as at the communication device user, can be used for analyzing breath sounds for detection of the breath alarm state. In this case, it was especially found that individual breaths can be identified from the breath sounds and as a result, a particular breath duration can be determined.

The determination of a sequence of breath durations makes possible different manners of analyzing the breathing of the communication device user. In this connection, it is always advantageous that a vital state of the communication device user can be inferred in a direct and reliable manner by the direct analysis of the breathing.

Due to the analysis of a sequence of breath durations, the additional analysis by the monitoring unit can be adapted individually to parameters of the breathing of the concrete communication device user.

The simple integration of the communication device according to the present invention into existing communication systems, for example, for firefighters is especially advantageous. The microphone unit and the output unit can thus be further used for an existing communication system, and the present invention can be implemented in a simple manner and without especially high production costs due to the provision of a suitable monitoring unit.

The communication device according to the present invention is already a communication device because it outputs the alarm output when the breath alarm state is present and thus communicates an alarm. The communication device is preferably also capable of processing speech or it is at least involved in the processing of speech, however, without this property being necessary for the communication device according to the present invention. Thus, the communication device is not configured for processing speech in some exemplary embodiments according to the present invention.

The units of the communication device according to the present invention may be arranged in a common housing or be arranged, for example, in different housings, which are at least partially arranged at a spaced location from one another.

The triggering of the breath alarm state describes the activation of a state, which indicates that the breathing of the communication device user is currently disturbed or will be disturbed in the near future.

The alarm output indicates the breath alarm state here because it indicates optically, acoustically and/or haptically that a breath alarm state is present and preferably also what type of breath alarm state is present.

The acoustic signal received typically comprises, in addition to the breath sounds to be analyzed, also ambient noises of the communication device user and possibly speech of the communication device user. The monitoring unit is preferably configured here to further process the acoustic signal such that the sounds of individual breaths and hence a particular breath duration can be determined based on a typical frequency response of a breathing.

Examples of amplitude- and/or frequency-dependent pattern recognition are known to the person skilled in the art. In particular, predefined breathing patterns and/or predefined patterns of known ambient noises can be compared with the pattern of the acoustic signal received, especially with a frequency spectrum of the acoustic signal received in order to make possible a splitting into breaths. For example, the acoustic analysis of breath sounds for detection of problems during breathing is described in DE 43 38 466 A1. Alternatives or additional examples of the analysis of breath sounds are known, in principle, to the person skilled in the art and will be explained in detail, for example, within the framework of the description of the figures.

The consecutive sequence of breath durations is based according to the present invention on at least two breath durations of two breaths. Consecutive breaths are preferably analyzed in this case. As an alternative or in addition, an average breath duration is determined via a predetermined number of breaths, in order to monitor the presence of the breath alarm state.

The breath alarm state is determined at least based on the sequence of breath durations according to the present invention. In addition, additional features, for example, the breath sounds, may form a basis for the determination of the breath alarm state, for example, the amplitude and/or frequency of breath sounds.

Preferred embodiments of the communication device according to the present invention will be described below.

In an especially preferred embodiment, the output unit is configured to output a wireless alarm output at an external device. The breath alarm state is especially advantageously outputted in this embodiment at a different position rather than directly at the communication device. Bystanders, for example, firefighters or a head of operations of a firefighting mission, can, as a result, be informed about the breath alarm state of a communication device user and possibly respond correspondingly. The wireless output can be provided, for example, via a Bluetooth connection, a WLAN connection, a BLE connection, a LoRa connection, a ZigBee connection or the like. Such wireless interfaces are known to the person skilled in the art and will therefore not be explained in detail below.

In another especially advantageous embodiment, the output unit has a speaker module and is configured to output an acoustic output in the surrounding area of the microphone unit via the speaker module. In this embodiment, the communication device user is especially advantageously informed about the presence of a breath alarm state. As a result of this, the communication device user may possibly still take precautions against an imminent shortness of breath. Thus, the communication device user may consciously calm down, leave a critical area of the mission site and/or possibly put down his/her oxygen supply. As an alternative or in addition to the information that a breath alarm state is present, the acoustic output may also have a meditative output, for example, a melody, to contribute to the calming down of the communication device user.

In another advantageous embodiment, the monitoring unit is further configured to determine a particular acoustic breath spectrum of a breath and to determine the breath alarm state as a function of a consecutive sequence of breath spectra. For example, an exhaled breath volume can, as is well known, be inferred from the breath spectrum. Consequently, in addition to the determined breath duration of a breath, additional information is available to detect a critical state of the breathing and hence a breath alarm state to be triggered.

In a preferred embodiment, the breath alarm state is triggered when a breath alarm type from a predefined group of breath alarm types is detected by the monitoring unit, the group of breath alarm types comprising: A frequency of breaths above an upper frequency threshold value; a frequency of breaths below a lower frequency threshold value; a time period since a last breath above a time threshold value; an amplitude or a sequence of amplitudes of a breath above an upper amplitude threshold value; and an amplitude or a sequence of amplitudes below a lower amplitude threshold value. Such breath alarm types lead in an especially reliable manner to the determination of the breath alarm state, since only a comparison with a threshold value has to be carried out. For example, an especially high respiration rate or an especially low respiration rate may thus indicate a situation that is critical for the health of the communication device user. In particular, a very low respiration rate may indicate that breathing will stop soon. Analogously, an especially high or an especially low amplitude of the breathing may likewise indicate a critical state of health.

In an especially preferred embodiment, the monitoring unit is further configured to determine an estimated breathing gas consumption for the period of time indicated by the consecutive sequence on the basis of the determined breath duration and of the determined breath spectrum of a particular breath for a past consecutive sequence of breaths on the basis of a stored predefined calculation rule and to trigger the breath alarm state as a function of the estimated breathing gas consumption. An especially high or especially low breathing gas consumption may indicate a critical state of health of the communication device user. Additional physiological variables of the communication device user may, in addition, be inferred from such physiological parameters. For example, the minute volume of the breathing and/or a changing state of the lung activity can thus be detected.

In an especially preferred variant of the above embodiment, the monitoring unit is further configured to compare a predefined amount of breathing gas with the estimated breathing gas consumption and to trigger the breath alarm state based on this comparison. This variant is especially advantageous for a communication device user, who at the same time has access to an external oxygen supply, for example, access to an oxygen cylinder. For such an external oxygen supply, an available amount of breathing gas can be determined and taken into consideration as a predefined amount of breathing gas. Thus, the comparison between the predefined amount of breathing gas and estimated breathing gas consumption shows whether breathing gas from the external oxygen supply is still available. In particular, the duration, for which an amount of breathing gas still currently available in the external oxygen supply is sufficient in view of the current breathing gas consumption can be inferred on the basis of the determined breathing gas consumption. Thus, the communication device user can be warned early if the external oxygen supply can no longer provide oxygen within a predefined time interval, especially within the next 10 minutes, especially preferably within the next 5 minutes. In this case, the breath alarm state may according to the present invention be both the state that the amount of oxygen is still only sufficient for 10 minutes, and the state that this amount of oxygen is sufficient only for 5 minutes, and the state that the predefined amount of breathing gas from the external oxygen supply is used up. In an especially preferred example of the embodiment according to the present invention, the alarm output indicates the still available duration, during which the communication device user is still being supplied by the external oxygen supply. The communication device user himself/herself and/or an external head of operations can, as a result, be informed about the critical state of the external oxygen supply.

In another especially preferred embodiment, the communication device according to the present invention has, furthermore, a speech processing unit, which is configured to receive the input signal and to process speech components in the acoustic signal received via the microphone unit and to output the acoustic signal as an output signal via the output unit or via an additional output unit. In this embodiment, the communication device is configured to also make possible, in addition to the processing of the breath sounds due to pattern recognition, a processing of the speech components from the received acoustic signal. As a result, the communication device may also be utilized for communication of a plurality of communication device users with one another and/or with a head of operations, for example, during a firefighting mission. The processing of speech components is known, in principle, to the person skilled in the art, so that this will not be discussed in detail below.

A communication system comprising a plurality of communication devices according to the above embodiments is provides according to a second aspect of the present invention for accomplishing the above-mentioned object. In this case, the respective communication devices are, furthermore, configured to communicate with one another via the output unit and via the additional output unit.

Such a communication system combines both the known advantages of the communication of the members of a rescue team, for example, firefighters, during a rescue and the advantageous monitoring of the breath sounds of the communication device user by the communication device according to the present invention. Thus, it is advantageously possible for any member of a rescue team involved in the rescue operation to automatically check whether the person is currently unconscious or will be unconscious in the near future and/or whether the external oxygen supply provided can no longer provide breathing gas in the next few minutes.

In addition, the communication system according to the present invention advantageously allows an output regarding the current breath alarm state directly at the communication device user and thus directly at the person in question.

In an advantageous embodiment, the communication system according to the present invention has, furthermore, a central management component, which is configured to receive the alarm output and to provide a communication connection (communication link) for the acoustic contacting of the corresponding communication device user with the communication device triggering the alarm output. The central management component is preferably operated and/or monitored by a head of operations of the mission. In addition to the output made possible by the communication device according to the present invention at the communication device user himself, a head of operations is therefore also informed about the breath alarm state. In this embodiment, the head of operations may, in addition, make contact with the communication device user in order to inform the same about additional steps to be carried out, for example, about an imminent termination of the mission. Unconsciousness, a shortness of breath or a similar critical state of health of the communication device user can, as a result, advantageously be prevented.

According to a third aspect of the present invention, a gas mask with a communication device according to at least one of the above embodiments is provided for accomplishing the above-mentioned object.

The gas mask according to the present invention is especially advantageous since the members of a rescue team using a respirator are exposed to an increased risk of breathing problems and/or oxygen supply problems. Consequently, the communication device according to the present invention is especially advantageous since such problems can be detected in an automated manner and then corresponding countermeasures can be triggered for ensuring the health of the communication device user. In an especially advantageous embodiment, the communication device within the gas mask is configured to determine the estimated breathing gas consumption and possibly to derive the breath alarm state from this. Thus, the comparison of the estimated breathing gas consumption with the predefined amount of breathing gas is especially advantageous for a person, who is wearing a gas mask and is thereby reliant on the external oxygen supply.

The provision of the communication device in a gas mask advantageously allows an arrangement of the microphone unit in the area of the mouth and/or of the nose of the communication device user. In addition, the arrangement of a speaker module in the area of the microphone unit, especially in the area of at least one ear of the communication device user, is possible.

According to a fourth aspect of the present invention, a helmet, especially a firefighter helmet, with a communication device according to at least one of the above embodiments is provided for accomplishing the above-mentioned object.

The integration of the communication device according to the present invention into a helmet and/or into a gas mask avoids the carrying along of additional devices, since a helmet and a gas mask would possibly be worn by the communication device user anyway, so that no additional device has to be put on in preparation, for example, of a mission.

The helmet makes possible especially advantageously the arrangement of a speaker module of the communication device in the area of at least one ear of the communication device user.

According to a fifth aspect of the present invention, a process for monitoring breathing is proposed for accomplishing the above-mentioned object, having the steps:
receipt of an acoustic signal, which indicates breath sounds of a communication device user of the communication device, from a surrounding area;
conversion of the acoustic signal into a digital input signal;
receipt of the digital input signal, splitting of the indicated breath sounds into individual breaths on the basis of an amplitude- and/or frequency-dependent pattern recognition and determination of a respective breath duration of a breath;
triggering of a breath alarm state of the communication device as a function of a consecutive sequence of breath durations; and
outputting of an alarm output indicating the breath alarm state after a triggering of the breath alarm state.

The process according to the present invention is carried out by the communication device according to the first aspect of the present invention and thereby comprises all the advantages of this communication device. The process according to the present invention allows especially an automated monitoring of the breath sounds of a communication device user. Consequently, it can be detected in an especially reliable manner when, for example, the breathing of this person stops suddenly and/or becomes unusually accelerated or slowed down.

The present invention shall now be explained in more detail on the basis of advantageous exemplary embodiments, which are schematically shown in the figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view of a communication device according to a first exemplary embodiment of a first aspect of the present invention;

FIG. 2 is a schematic view of a communication device according to a second exemplary embodiment of the first aspect of the present invention;

FIG. 3 is a diagram for explaining a pattern recognition for indicated breath sounds according to the first aspect of the present invention;

FIG. 4 is a schematic view of a communication system according to an exemplary embodiment of a second aspect of the present invention;

FIG. 5 is a schematic view of a gas mask according to an exemplary embodiment of a third aspect of the present invention;

FIG. 6 is a schematic view of a helmet, especially a firefighter helmet, according to an exemplary embodiment of a fourth aspect of the present invention; and

FIG. 7 is a flow chart of a process according to an exemplary embodiment of a fifth aspect of the present invention.

### DESCRIPTION OF PREFERRED EMBODIMENTS

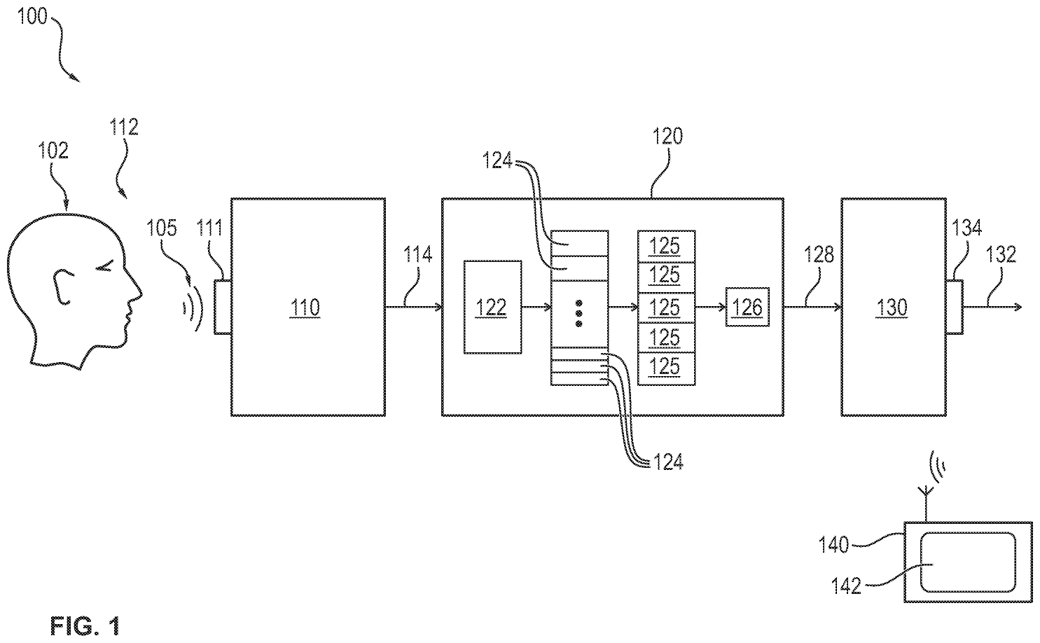

Referring to the drawings, FIG. 1 shows a schematic view of a communication device 100 according to a first exemplary embodiment of a first aspect of the present invention.

The communication device 100 is configured for monitoring the breathing of a communication device user 102. The communication device 100 has for this a microphone unit 110, a monitoring unit 120 and an output unit 130.

The microphone unit 110 is configured to receive an acoustic signal 105, which indicates the breath sounds of a communication device user 102 of the communication device 100, from a surrounding area 112 of the microphone unit 110, and to convert the acoustic signal into a digital input signal 114. The receipt is carried out in the present case via a microphone interface 111. In this case, the acoustic signal 105 indicates preferably all sounds and noises, which are present in the surrounding area 112 of the microphone unit 110. The microphone unit 110 is therefore preferably arranged in the immediate vicinity in space of the nose and/or of the mouth of the communication device user 102. The conversion of the acoustic signal 105 by the microphone unit 110 comprises according to the present invention a digitization of the acoustic signal 105 into the digital input signal 114.

The monitoring unit 120 is configured to receive the digital input signal 114. The received digital input signal 114 is then further processed on the basis of an amplitude- and/or frequency-dependent pattern recognition 122 such that the indicated breath sounds are split into individual breaths 124. The particular breath duration 125 of a breath 124 can be determined on the basis of these breaths 124. In this case, the monitoring unit 120 is further configured to trigger a breath alarm state 126 of the communication device 100 as a function of a consecutive sequence of breath durations 125. The monitoring unit 120 is in this case at least configured to distinguish between a state, in which no breath alarm state 126 is present and a state, in which the breath alarm state 126 is present. The monitoring unit 120 preferably also detects the present breath alarm type and outputs this together with the information about the presence of the breath alarm state 126.

For this, the communication device 100 has the output unit 130, which is configured to output an alarm output 132 indicating the breath alarm state 126 after a triggering of the breath alarm state 126. For this, the monitoring unit 120 outputs an alarm state signal 128 at the output unit 130. The alarm state signal 128 indicates that a breath alarm state 126 was detected. The alarm state signal preferably also indicates a present breath alarm type.

In the exemplary embodiment being shown, the alarm output 132 is carried out via an interface 134. This interface 134 is configured for the output of a wireless signal, for example, a Bluetooth, WLAN, ZigBee or LoRa signal. In the exemplary embodiment shown, this alarm output 132 is received via an external device 140 with a visual output 142 and is outputted visually and/or acoustically. In this case, the signal received from the external device 140 may be a signal triggered by an additional device, not shown, based on the alarm output 132.

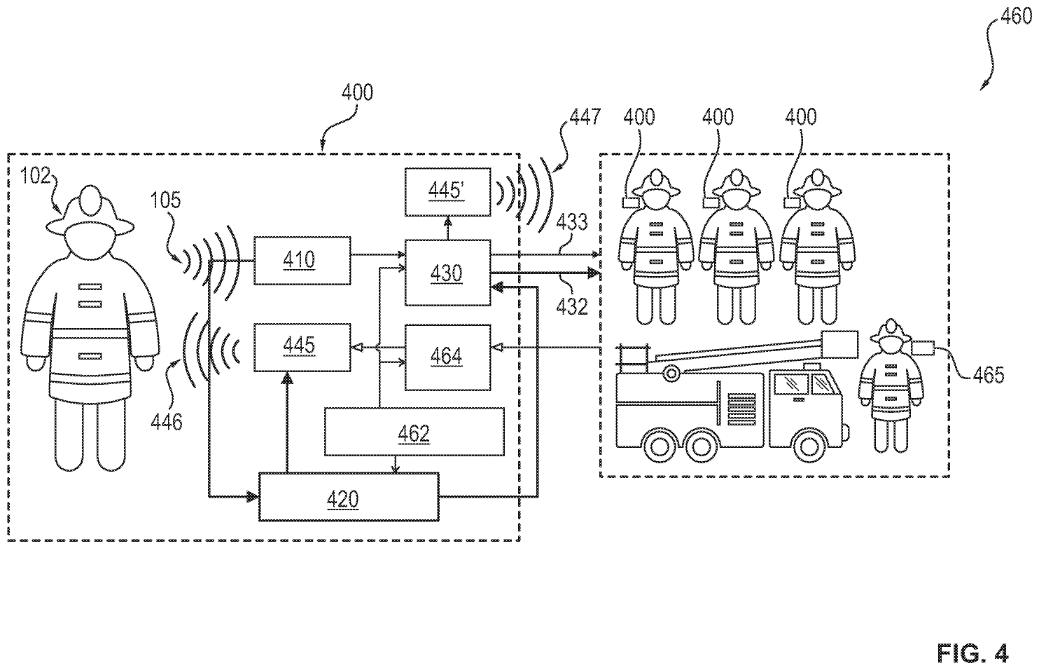

All units of the communication device shown are preferably arranged in a common housing, which is not shown. As an alternative, at least some units may be arranged in a separate housing. The communication device 100 is especially preferably part of a communication system, as it is shown in FIG. 4.

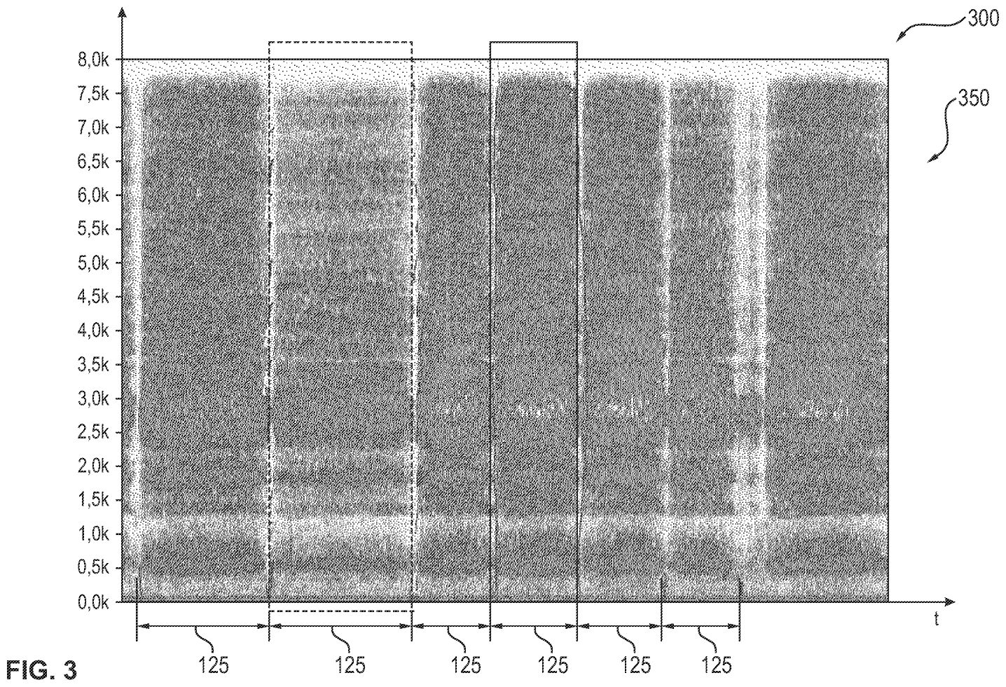

The analysis of the digital input signal 114 by the monitoring unit 120 is described more precisely within the framework of FIG. 3. In this regard, FIG. 1 only shows that the last five breath durations 125 are analyzed in order to determine whether a breath alarm state 126 is present. The sequence of breath durations according to the present invention only has to comprise at least two breath durations.

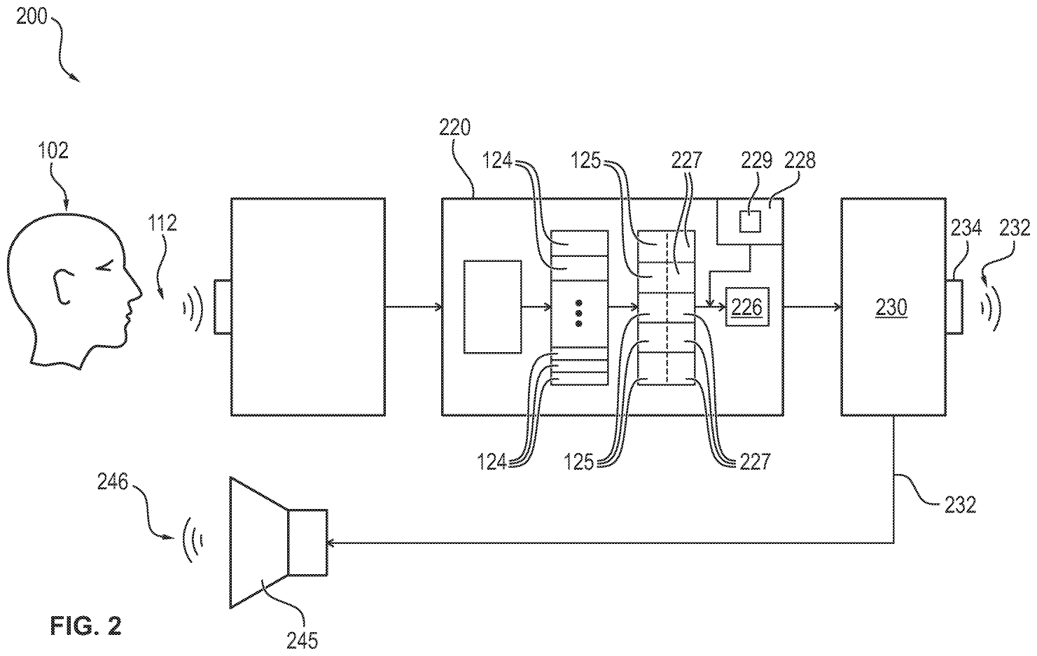

FIG. 2 shows a schematic view of a communication device 200 according to a second exemplary embodiment of the first aspect of the present invention.

The communication device 200 differs from the communication device 100 shown in FIG. 1 by the alarm output 232 being carried out via the interface 234 as a wireless output at an additional external device, not shown, and additionally being carried out via a cable-based connection to a speaker module 245 of the communication device 200. The speaker module 245 forms in this case a part of the output unit 230. The speaker module 245 is configured to provide an acoustic output 246 in the surrounding area 112 of the communication device carrier 102. The communication device user 102 is, as a result, informed about the presence of the breath alarm state 126. As a result, corresponding actions can be taken by the communication device user 102 or by persons in his/her surrounding area, in order to prevent a worsening of the state of health of the communication device user 102.

Furthermore, the communication device 200 differs by the monitoring unit 220 being further configured to determine a respective acoustic breath spectrum 227 of a breath 124 and to determine the breath alarm state 226 as a function of a consecutive sequence of breath spectra 227 and breath durations 125. In this case, the breath alarm state is triggered when a breath alarm type is detected from a predefined group of breath alarm types. The group of breath alarm types comprises at least: A frequency of breaths above an upper frequency threshold value; a frequency of breaths below a lower frequency threshold value; a time period since a last breath above a time threshold value; an amplitude or a sequence of amplitudes of a breath above an upper amplitude threshold value; and an amplitude or a sequence of amplitudes below a lower amplitude threshold value.

The time threshold value is preferably at most 15 seconds, and especially at most 12 seconds. The upper frequency threshold value is preferably at least 22 breaths per minute, especially at least 30 breaths per minute. The lower frequency threshold value is preferably at most 7 breaths per minute, and especially at most 5 breaths per minute.

As an alternative or in addition to the provision of predefined threshold values, a relative change of the frequency and/or of the amplitude of breaths may lead to the detection of a breath alarm state. Thus, a marked relative change of these variables may indicate a worsening of the health situation of the communication device user.

The respective threshold values are stored in an internal memory 228 of the monitoring unit 220 in the present case. In addition to the threshold values, a value for a predefined amount of breathing gas 229 is also stored in the internal memory 228. The predefined amount of breathing gas 229 indicates in the present exemplary embodiment how much breathing gas is provided by an external oxygen supply, not shown, to the communication device user 102 during a mission.

The monitoring unit 220 is, in addition, configured to determine an estimated consumption of breathing gas for the period of time indicated by the consecutive sequence on the basis of the determined breath duration 125 of the determined breath spectrum 227 of a particular breath 124 for a past consecutive sequence of breaths on the basis of a predefined calculation rule stored in the internal memory 228.

The determination of the breathing gas consumption makes possible a comparison with the stored predefined amount of breathing gas 229 and therefore an estimation of the point in time at which oxygen can no longer be made available to the communication device user 102 by the external oxygen supply. Therefore, in this exemplary embodiment, in addition to the warning that the breath sounds indicate a current or imminent health-related problem, a problem with regard to a provided oxygen supply may also be detected, so that the communication device user 102 and/or an external head of operations can take this problem into consideration early. In this case, the alarm output 232 preferably indicates an estimated period of time, after which the communication device user 102 will probably no longer receive oxygen by the provided oxygen supply.

FIG. 3 shows a diagram 300 for explaining a pattern recognition for indicated breath sounds 350 according to the first aspect of the present invention.

The diagram 300 shows a spectrogram of the breath sounds 350, which were recorded with the communication device according to the present invention, for example, with the communication device 200 from FIG. 2. This recording of the breath sounds 350 can be detected according to the present invention continuously and in real time by the corresponding microphone unit and be processed via the corresponding monitoring unit. In this case, the diagram 300 shows the time in the direction of the X axis and the amplitude of certain frequency bands in the direction of the Y axis, which is indicated in the present view by a density of dots, wherein a high dot density corresponds to a high amplitude and a low dot density corresponds to a low amplitude.

The air consumption of the communication device user can be inferred by the loudness of the breath sounds 350 and the duration of the inhalation and thus of the breath. A sequence of consecutive breaths with different breath durations 125 is shown the diagram 300. The frame shown in broken lines shows an easy inhalation based on the low dot density and hence based on the low amplitude, whereas the frame shown in solid lines with a high dot density and hence with a high amplitude shows a deep inhalation. The spectrogram can be correspondingly analyzed by the predefined type of pattern recognition, so that the air consumption can be inferred from the duration and intensity of a corresponding breath. The basis for this may be an empirically determined function that describes the connection between the intensity and duration of a breath and the air volume flowing through in this case. As an alternative to an empirically determined function, value tables or similar known assignment rules can allow the analysis of the air consumption on the basis of the duration and the intensity of a particular breath.

The diagram 300 also clearly shows that the breath duration 125 can be detected reliably by the automated detection of such spectrographic information via the monitoring unit. Thus, the analysis of the breath sounds according to the present invention within the received acoustic signal may indicate in a simple and reliable manner the current health situation of the communication device user and preferably even indicate the volume of oxygen consumed up to now during a mission.

The presence of a spectrogram according to FIG. 3 makes possible, in addition, the recognition of a breathing pattern, which may represent an indication of the presence of a breath alarm state. For example, exhaustion, a starting panic reaction or impending unconsciousness may thus be indicated by an especially high amplitude of a breath, by an especially low amplitude of a breath and/or by an especially high frequency of breaths.

FIG. 4 shows a schematic view of a communication system 460 according to an exemplary embodiment of a second aspect of the present invention.

The communication system 460 comprises a plurality of communication devices 400, which are configured to communicate with one another via the corresponding output unit 430.

Only one communication device 400 from the plurality of communication devices is shown in detail as an example.

In addition to the microphone unit 410, the monitoring unit 420 and the output unit 430, which all essentially function as was explained within the framework of FIG. 2 on the basis of the communication device 200, the communication device 400 has additional units, which will be explained below.

Within the framework of the above exemplary embodiments, the necessary power supply unit 462 was not shown for the sake of clarity. The power supply unit 462 is connected both to the monitoring unit 420 and to the receiving amplifier 464 and to the output unit 430. The output unit 430 additionally functions in the exemplary embodiment shown as an amplifier of acoustic signals 105 received via the microphone unit 410. In the exemplary embodiment shown, the output unit 430 provides a schematically shown communication with the additional communication devices 400 of the communication system 460. In addition, a communication with a central management component 465 of the communication system 460 is provided. The communication devices 400 are arranged in the surrounding area of the mouth and/or of the nose of a member of a rescue team, of a firefighter in the present case, whereas the central management component 465 is monitored by a head of operations.

The central management component 465 is configured to receive the alarm output 432 and to provide a communication connection with the communication device 400 triggering the alarm output 432 for the acoustic contacting of the corresponding communication device user 102. This contacting is carried out in the present case via the receiving amplifier 464, which passes on a correspondingly digitized signal to a first speaker module 445, which then leads to an acoustic output 446 in the surrounding area of the microphone unit 410.

In addition, a speech processing unit, which is configured to receive the digital input signal from the microphone unit 410 and to process the speech components from the acoustic signal 105 and to output same via the corresponding output signal 433, is located within the output unit 430.

Finally, the communication device 400 has a second speaker module 445′, which is oriented in a direction away from the communication device user 102, and which is configured to output an alarm generation sequence of sounds 447 to be provided via the output unit 430 if the breath alarm state determined by the monitoring unit 420 requires this. The alarm generation sequence of sounds 447 indicates to bystanders that a breath alarm state is present at the communication device user 102. In the exemplary embodiment shown, the alarm generation sequence of sounds is provided by the monitoring unit 420 via the output unit 430 for the second speaker module 445′, which outputs the alarm generation sequence of sounds 447. The monitoring unit in an alternative or additional exemplary embodiment is connected directly to the second speaker module, wherein the second speaker module and/or the first speaker module are parts of the output unit according to the present invention.

By contrast to the alarm generation sequence of sounds 447, the acoustic output 446 may, as an alternative or in addition to the breath alarm state or to a breath alarm type, also comprise a calming sequence of sounds, for example, a calming announcement and/or a calming music. As is known, such a sequence of sounds forms an active psychological support in a stress situation. As a result, a stress level of the communication device user 102 can be lowered and hence a respiration rate can be reduced. In case of irregularities in breathing that have occurred only because of the stress level, this may already lead to a more regular breathing and hence to the detection of a no longer present breath alarm state. If a breath alarm state is no longer detected by the monitoring unit 420, the corresponding alarm output is no longer outputted and/or an output is carried out that the previous breath alarm state is currently no longer present.

Should the stopping of breathing be detected, the communication device 400 shows the shape of a dead man warning according to the present invention. Thus, a corresponding breath alarm state is triggered in case of a too long breath duration, which would be detected by the monitoring unit, and a corresponding alarm output is outputted in order to inform the head of operations and bystanders about the stopping of the breathing.

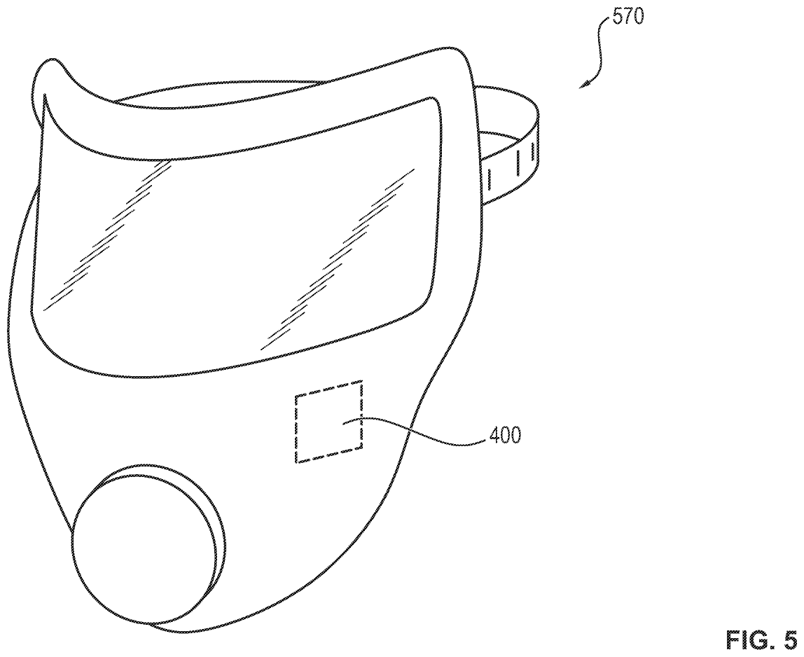

FIG. 5 shows a schematic view of a gas mask 570 according to an exemplary embodiment of a third aspect of the present invention.

The gas mask 570 according to the present invention has the communication device 400 according to the present invention. The configuration of the communication device 400 is described within the framework of FIG. 4. The plurality of gas masks with the same communication device 400, which together with the shown communication device 400 form the communication system 460 according to FIG. 4, are not shown. The communication device 400 is preferably permanently integrated into the gas mask 570. The microphone unit is in this case arranged in the area of the mouth and/or of the nose of the communication device user.

The communication device according to the present invention is especially advantageous for uses of a gas mask, because the breathing within the gas mask is, in principle, more difficult than without a restriction of the available breathing gas. Therefore, the precise monitoring of the breath sounds is advantageous for people with breathing that is currently difficult anyway, for example, for the users of a gas mask. The monitoring unit of the communication device 400 especially preferably detects, on the basis of the received breath sounds, the estimated breathing gas consumption and compares this with a predefined amount of breathing gas provided via an external oxygen supply. As a result, the communication device according to the present invention makes possible in the exemplary embodiment shown an alarm generation in case the available amount of oxygen falls below a critical time-dependent and/or volume-dependent threshold value.

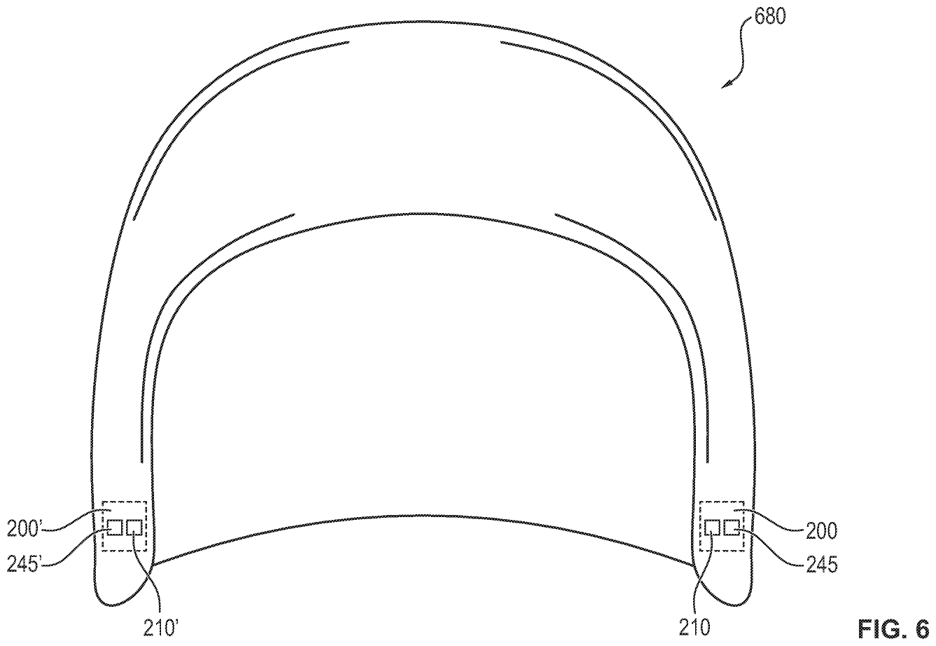

FIG. 6 shows a schematic view of a helmet 680, especially of a firefighter helmet, according to an exemplary embodiment of a fourth aspect of the present invention.

The helmet 680 according to the fourth aspect of the present invention comprises in the present case the communication device 200, which was explained within the framework of FIG. 2. The communication device is comprised of a first part 200 and of a second part 200′. In this case, the microphone unit 210, 210′ has two separate parts, which are arranged on the left-hand side and on the right-hand side of the face of the communication device user at the helmet 680. In addition, the speaker module 245, 245′ has two separate parts, which are arranged at the helmet 680 on the left-hand side and on the right-hand side of the face of the communication device user.

Due to the provision of a plurality of parts, the breathing of the communication device user is especially reliably detected by the microphone unit. In addition, the provision of two parts for the speaker module ensures an especially reliable and pleasant output of the sounds to be outputted.

The exemplary embodiment shown illustrates that the communication device according to the present invention may be arranged in a plurality of separate housings. The communication device according to the present invention may especially be arranged at positions arranged at a spaced location from one another in relation to the communication device user, for example, at positions arranged at a spaced location within a gas mask and/or at positions arranged at a spaced location within a helmet.

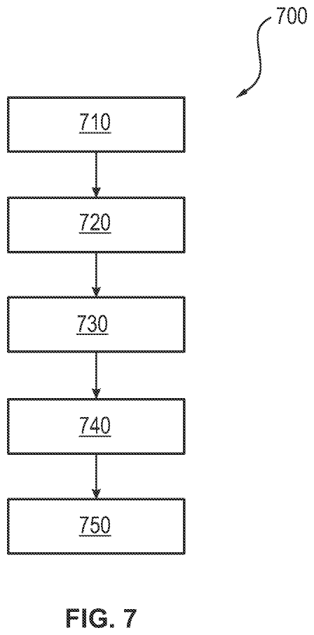

FIG. 7 shows a flow chart of a process 700 according to an exemplary embodiment of a fifth aspect of the present invention.

The process 700 according to the present invention is configured for monitoring breathing. In this case, the process 700 has the steps described below.

A first step 710 comprises a receipt of an acoustic signal, which indicates the breath sounds of a communication device user of the communication device, from a surrounding area.

A next step 720 comprises a conversion of the acoustic signal into a digital input signal.

A next step 730 comprises a receipt of the digital input signal, a splitting of the indicated breath sounds into individual breaths on the basis of an amplitude-dependent pattern recognition and/or frequency-dependent pattern recognition and a determination of a respective breath duration of a breath.

A further step 740 comprises a triggering of a breath alarm state of the communication device as a function of a consecutive sequence of breath durations.

A final step 750 comprises an outputting of an alarm output indicating the breath alarm state after the triggering of the breath alarm state.

The steps of the process 700 according to the present invention are also carried out consecutively. Here, the receipt of the acoustic signal according to step 710 is preferably carried out continuously or at least essentially continuously in order to ensure an analysis of the indicated breath sounds that is as reliable as possible. Correspondingly, steps 720 and 730 are also carried out essentially continuously. The processing in real time or at least essentially in real time is essential for the present invention, since otherwise a warning regarding the breath alarm state cannot make it possible any longer to take countermeasures for the protection of the communication device carrier.

Steps 740 and 750 are only carried out when a breath alarm state is present and a corresponding output therefore has to be carried out according to the present invention.

In an additional exemplary embodiment, the outputting of the alarm output according to step 750 is carried out only after a brief validation of the breath alarm state triggered within the framework of step 740. Such a validation may be, for example, a rechecking of the indicated breath sounds according to the predefined amplitude- and/or frequency-dependent pattern recognition.

The process according to the present invention was preferably carried out during the duration of a mission of the communication device user. This corresponds to approximately a time of less than 4 hours, preferably less than 2 hours in the field of firefighting missions.

While specific embodiments of the invention have been shown and described in detail to illustrate the application of the principles of the invention, it will be understood that the invention may be embodied otherwise without departing from such principles.

### LIST OF REFERENCE NUMBERS

100, 200, 200′, 400 Communication device
102 Communication device user
105 Acoustic signal
110, 210, 210′, 410 Microphone unit
111 Microphone interface
112 Surrounding area of the microphone unit
114 Digital input signal
120, 220, 420 Monitoring unit
122 Amplitude- and/or frequency-dependent pattern recognition
124 Breath
125 Breath duration
126, 226 Breath alarm state
128 Alarm state signal
130, 230, 430 Output unit
132, 232, 432 Alarm output
134, 234 Interface
140 External device
142 Visual output
227 Breath spectrum
228 Internal memory
229 Predefined amount of breathing gas
245, 245′ Speaker module
246, 446 Acoustic output
300 Diagram
350 Breath sounds
433 Output signal
445 First speaker module
445′ Second speaker module
447 Alarm generation sequence of sounds
460 Communication system
462 Power supply unit
464 Receiving amplifier
465 Central management component
570 Gas mask
680 Helmet
700 Process
710, 720, 730, 740, 750 Process steps

What is claimed is:

1. A communication device for monitoring breathing, the communication device comprising:
a microphone unit, which is configured to receive an acoustic signal, which indicates breath sounds, of a communication device user of the communication device, from a surrounding area of the microphone unit, and to convert the acoustic signal into a digital input signal;
a monitoring unit, which is configured to receive the digital input signal and to split the indicated breath sounds into individual breaths based on an amplitude-dependent pattern recognition or a frequency-dependent pattern recognition or on an amplitude and frequency-dependent pattern recognition and to determine a particular breath duration of a breath and further configured to trigger a breath alarm state of the communication device as a function of a consecutive sequence of breath durations; and
an output unit, which is configured to output an alarm output indicating the breath alarm state after a triggering of the breath alarm state.

2. A communication device in accordance with claim 1, wherein the output unit is configured to output a wireless alarm output at an external device.

3. A communication device in accordance with claim 1, wherein the output unit comprises a speaker module and is configured to output an acoustic output in the surrounding area of the microphone unit via the speaker module.

4. A communication device in accordance with claim 1, wherein the monitoring unit is further configured to determine a particular acoustic breath spectrum of a breath and to determine the breath alarm state as a function of a consecutive sequence of breath spectra.

5. A communication device in accordance with claim 1, wherein:
the breath alarm state is triggered when a breath alarm type from a predefined group of breath alarm types is recognized by the monitoring unit; and
the group of breath alarm types comprises: a frequency of breaths above an upper frequency threshold value; a frequency of breaths below a lower frequency threshold value; a time period since a last breath above a time threshold value; an amplitude or a sequence of amplitudes of a breath above an upper amplitude threshold value; and an amplitude or a sequence of amplitudes below a lower amplitude threshold value.

6. A communication device in accordance with claim 4, wherein the monitoring unit is further configured to determine an estimated breathing gas consumption for a period of time indicated by the consecutive sequence based on the determined breath duration and of the determined breath spectrum of a particular breath for a past consecutive sequence of breaths based on a stored predefined calculation rule and to trigger the breath alarm state as a function of the estimated breathing gas consumption.

7. A communication device in accordance with claim 6, wherein the monitoring unit is further configured to compare a predefined amount of breathing gas with the estimated breathing gas consumption and to trigger the breath alarm state based on the comparison.

8. A communication device in accordance with claim 1, further comprising a speech processing unit, which is configured to receive the input signal and to process speech components into the acoustic signal received via the microphone unit and to output same as an output signal via the output unit or via an additional output unit.

9. A communication device in accordance with claim 8, further comprising at least another communication device to provide a plurality of communication devices, wherein the respective communication devices are further configured to communicate with one another via the output unit and via the additional output unit.

10. A communication device in accordance with claim 9, further comprising a central management component, which is configured to receive the alarm output and to provide a communication connection for an acoustic contacting of the corresponding communication device user with the communication device triggering the alarm output.

11. A communication device in accordance with claim 1, in combination with a gas mask, wherein the communication device is connected to the gas mask.

12. A communication device in accordance with claim 1, in combination with a helmet, wherein the communication device is connected to the helmet.

13. A process for monitoring breathing, the process comprising the steps of:
receiving an acoustic signal, which indicates breath sounds of a communication device user of the communication device, from a surrounding area;
converting the acoustic signal into a digital input signal;
receiving the digital input signal, splitting of the indicated breath sounds into individual breaths on the basis of an amplitude-dependent pattern recognition or a frequency-dependent pattern recognition or on an amplitude and frequency-dependent pattern recognition and determining a respective breath duration of a breath;
triggering a breath alarm state of the communication device as a function of a consecutive sequence of breath durations; and
outputting an alarm output indicating the breath alarm state after a triggering of the breath alarm state.
